(19) United States
(12) Patent Application Publication
(10) Pub. No.: US 2008/0156068 A1
(43) Pub. Date: Jul. 3, 2008

(54) Treatment of Chromosomal Abnormalities in Fetuses Through A Comprehensive Metabolic Analysis of Amniotic Fluid

(76) Inventor: Paddy Jim Baggot, Los Angeles, CA (US)

Correspondence Address: LYON & LYON LLP, 633 WEST FIFTH STREET, SUITE 4700, LOS ANGELES, CA, 90071, US

(21) Appl. No.: 12/048,078

(22) Filed: Mar. 13, 2008

Related U.S. Application Data
(63) Continuation of application No. 09/499,006, filed on Feb. 4, 2000, now abandoned.

(51) Int. Cl. G01N 30/02; G01J 3/28

(52) U.S. Cl. 73/19.02; 356/326

(57) ABSTRACT

A specimen of an amniotic fluid is obtained and analyzed by GC/MS in order to generate a comprehensive metabolic profile. The profile is analyzed by comparing the levels of metabolites with normal levels of those compounds. Specific treatment is then prescribed for the metabolite levels that differ from the norm. These metabolites that are present in different levels than a normal specimen may be indicative of chromosomal abnormalities such as Down Syndrome. The method of the present invention is used to model the complex problem of a chromosomal abnormality as the sum of several simpler problems that may be treatable. The comprehensive metabolic profile is used to detect chromosomal abnormalities, to suggest treatments for fetal chromosomal abnormalities, and to monitor their effectiveness.

### RELATED INFORMATION

This application is a continuation of co-pending application Ser. No. 09/499,006 filed on Feb. 4, 2000 and incorporated herein by reference.

### FIELD OF THE INVENTION

The present invention relates to the diagnosis of biochemical abnormalities found in a fetus. Specifically, the present invention is a comprehensive metabolic profile of an amniotic fluid specimen to diagnose and prescribe treatment for chromosomal disorders, preferably Down's Syndrome, that may be present in a fetus.

### BACKGROUND

Chromosomal abnormalities are forms of birth defects that occur in 2 to 3 out of every 1000 births. Chromosome abnormalities may involve duplications or defects in a whole chromosome or a portion of one or more chromosomes. The most common chromosome abnormality is Down Syndrome. In Down Syndrome, each cell contains three copies of chromosome number 21, a condition referred to as trisomy 21. A result of trisomy 21 is a 50% increase in gene dosage for each gene on chromosome 21. At least some of the abnormalities seen in Down Syndrome can be attributed to excessive gene dosage. Down Syndrome is a complicated disorder, affecting many aspects of physiology. Since affected individuals all have mental retardation, it is a significant disability.

Less common chromosomal disorders involve other chromosomes. Some chromosomal disorders result from a deletion or a duplication of a portion of a chromosome. Other chromosomal disorders result from an absence of an entire chromosome. For example, an absence of the X chromosome results in Turner Syndrome, referred to as monosomy X.

The relationship between chromosome abnormalities and physical or metabolic disorders in children is not always clear. Down Syndrome is the most common and most thoroughly studied chromosome abnormality. This has spurred some authors to look for treatable or correctable abnormalities in children with Down Syndrome. Le Jeune suggested that abnormalities of purine synthesis, thyroid metabolism, and perturbations of enzymes, specifically copper/zinc superoxide dismutase, phosphofructokinase, and cystathione beta synthase, were related to Down Syndrome. Le Jeune, J., Pathogenesis of Mental Deficiency in Trisomy 21, Am. J. Med. Genetics Suppl. 7: 20-30 (1990). Additionally, Patterson and Ekvall suggested that a number of vitamins, minerals, and other metabolic abnormalities contributed to Down Syndrome. Patterson, B. and Ekvall, S. W., Down Syndrome in Pediatric Nutrition in Chronic Diseases and Developmental Disorders: Prevention Assessment and Treatment, Oxford Univ. Press, New York, 149-156 (1993). These theories have not been confirmed by other authors.

Several treatments have been proposed to address Down Syndrome. Turkel advocated the use of “orthomolecular” therapy. Turkel, H. B., Medical Amelioration of Down Syndrome Incorporating the Orthomolecular Approach, Psychiatry 4(2): 102-115 (1975). Harrell, et al. suggested supplementation with megadose vitamins. Harrell, et al., Can Nutritional Supplements Help Mentally Retarded Children?, PNAS USA 78(1): 574-578 (1981). As reported by Patterson and Ekvall, attempts to verify the effectiveness of the aforementioned treatments have proven unsuccessful. The difficulty of verifying the effectiveness of proposed treatments is compounded since researchers have not confirmed many of the proposed abnormalities. Also, some abnormalities could interact with other abnormalities. Folate metabolism, for example, is also involved in purine synthesis. Consequently, there is a need for a method to comprehensively analyze a wide panel of metabolites in order to create a complete biochemical picture of a given chromosomal abnormality. Using this method, corresponding treatment may then be identified and prescribed as a result of the complete biochemical picture.

The treatments of Turkel and Harrell et al. might be the right treatment given at the wrong time. Treatment, to be effective, oftentimes must be administered at the proper time. For example, supplementation of folate before and during neural tube development often prevents spina bifida. However, folate supplementation given after birth would be ineffective in preventing spina bifida.

In particular, neurologic development proceeds sequentially. The proper development of a structure depends on previous development of prior structures. Once a structure has developed incorrectly, the error is fixed and cannot later be revised.

For example, the harmful effects of Down Syndrome on brain development begin before birth. A baby with Down Syndrome already has many characteristic features of the disease at the time of birth. There is often hypoplasis of the frontal lobes and cerebellum resulting in a reduced anteroposterior diameter of the head. This is commonly referred to as brachycephaly. The most important events in brain development are the earliest ones, and later developments are less important. As a result, there is a need for a method by which an abnormality, particularly a chromosomal abnormality, may be detected at any early stage of development, such as at the prenatal stage. A course of treatment may then be prescribed at a time that is beneficial to the baby.

In abnormal fetal development, as in other pathologies, existing techniques can frequently identify a deficiency or abnormality in the existence or metabolism of a physiologically significant species. For example, various methods for analysis of target analytes generally, and evaluation of analytes in regards to prenatal testing specifically, have been proposed. Examples of some of these methods are disclosed in the following U.S. Pat. Nos.: 5,326,708, 5,438,017, 5,439,803, 5,506,150, 5,532,131, and 5,670,380.

However, many vitamins are cofactors for multiple enzymes. Similarly, minerals can be cofactors for enzymes. A given metabolite might be a substrate in some reactions and/or a product in other reactions. A determination that a single metabolite, or a single vitamin or mineral is under or over-expressed in a particular pathology is often insufficient to provide a meaningful opportunity for therapeutic intervention. By comparison, comprehensive metabolic profiling would allow the physicians to see the whole panorama of metabolism and would allow an integrated, comprehensive characterization of the problem as well as an integrated, comprehensive approach to treatment.

Accordingly, there is a need for a method to comprehensively profile the metabolic abnormalities found in the fetus with Down Syndrome or other chromosome abnormalities. Ideally, a global screen should be performed for a wide range of metabolites at one time. The results of the global screen could then be utilized to construct a biochemical profile that suggests treatment pathways for possible fetal chromosomal abnormalities including Down Syndrome.

### SUMMARY OF THE INVENTION

The present invention uses a sample of amniotic fluid to generate a comprehensive metabolic profile to diagnose chromosomal abnormalities in the fetus. The profile can be generated by several analytical techniques, and the components of the profile can be varied based on the clinical indication of interest. For example, a procedure similar to one described by Shoemaker and Elliott can be used to screen a specimen of amniotic fluid for metabolites. Shoemaker and Elliott, Automated screening of urine samples for carbohydrates, organic and amino acids after treatment with urease, Journal of Chromatography, 562 (1991) 125-138, specifically incorporated herein by reference. A specimen of amniotic fluid is first obtained. For example, amniotic fluid may be taken from around the fetus during pregnancy. The specimen then is analyzed in a gas chromatograph/mass spectrophotometer (GC/MS).

The results of the GC/MS analysis are then used to generate the profile of the metabolites previously identified. The sample profile is compared with a control profile of metabolites that is representative of the normal levels of those metabolites. By analyzing the sample profile with respect to the normal profile, each metabolite that has a different level when compared with the normal level of that metabolite can be identified. Using the identified metabolites that have different values than the norm, a biochemical treatment may be prescribed that addresses the concentration, i.e., to increase or decrease, of each of those metabolites or the metabolism or physiology thereof. Using this method, an improved treatment for chromosomal abnormalities such as Down Syndrome may be prescribed by taking into account metabolic deficiencies on a global level rather than individually or in small groups.

### DETAILED DESCRIPTION OF THE INVENTION

As previously discussed, a procedure similar to the procedure described by Shoemaker and Elliott may be used to screen an amniotic fluid specimen for metabolites. An amniotic fluid specimen is obtained from the fetus to be evaluated. For example, amniotic fluid may be obtained by placing a needle through the abdomen and uterine wall into the uterus and withdrawing the fluid with a syringe. It is preferable that the syringe not be lubricated with glycerol. The fluid may be separated from any unwanted cells by centrifugation.

If storage of the specimen is required, the specimen is preferably maintained at −20° C. Subsequently, the specimen is transferred to a metabolic screening laboratory where a GC/MS apparatus is located. If the laboratory is remotely located, the specimen is shipped on dry ice. The specimen is then thawed in the laboratory in preparation for the test.

The specimen is injected into the GC/MS apparatus in order to identify and measure the metabolites. All chemical constituents of the specimen are separated by their GC retention times. The identity and quantity of each chemical constituent is then determined by the MS. The MS preferably sweeps through all masses from 15-650 Daltons every two seconds.

A comprehensive metabolic profile is then generated that indicates the identity and quantity of each metabolite. From these levels, the activity of the relevant enzymes related to each metabolite can be inferred. The metabolites included in the report may include, but are not limited to, organic acids, amino acids, glycine conjugates, fatty acids, vitamins, neurotransmitters, drugs, drug metabolites, hormones, and carbohydrates.

The levels of the metabolites for the sample are then compared with the representative, normal level for each. The levels may be compared by examining mean levels and standard deviations for each metabolite. Alternatively, the levels may be compared using medians and a nonparametric analysis. By analyzing the relative levels of the metabolites as compared with the normal levels, a suitable biochemical treatment may be prescribed for a particular condition.

Most of the metabolites are indicator substrates. An indicator substrate is a substrate that is metabolized by an enzyme. When the enzyme is deficient or its activity is low, the indicator substrate accumulates. When the enzyme activity is high, the metabolite may be low. If the activity of an enzyme is low, vitamin or mineral cofactors can be supplemented. If the activity of an enzyme is too high, it can be blocked or cofactors withheld. Some metabolites are vitamins or vitamin derivatives. If they are low, they can similarly be supplemented.

Any metabolite that may be included in a GC/MS report or analogous analytical technique may be analyzed by the method of the present invention. This method is also used to analyze a global metabolic profile to provide the biochemical equivalent of a complete physical exam. The method detects abnormalities through the comprehensive metabolic profiling which identifies multiple metabolic abnormalities. Each one of these abnormalities, when present by itself in a severe form, would cause mental retardation. Abnormalities such as Down Syndrome may then be modeled as the sum of a group of simpler abnormalities that may have an identifiable metabolic profile. Many of these simpler abnormalities may have treatments that are suggested by the results of the comprehensive metabolic profiling. Using the results of the comprehensive metabolic profile, chromosomal abnormalities may be identified and proper treatment may be suggested at an opportune time.

A key advantage of the method is the characterization of a disease state, a physiologic influence, or the effects of a drug. If a group of normal patients and a group of disease patients are characterized, the differences between the groups will form a biochemical characterization of the disease.

### Example Profile Analysis

To diagnose a fetus for chromosomal abnormalities using the method of the present invention, a metabolic profile must first be generated that is representative of the metabolite levels in an average patient suffering from the chromosomal abnormality that is to be diagnosed. Using the aforementioned GC/MS procedure, a metabolic profile for a group of 23 Down Syndrome patients was generated. The following tables represent the Down Syndrome metabolic profile separated into different metabolite groups. The tables also include results for a group of 41 normal patients, generated by the GC/MS procedure, for comparison purposes. Since the comparison data for a chromosomal abnormality may be analyzed by examining either the mean levels of the metabolites or the median levels of the metabolites, two tables are presented for each metabolite grouping with one table presenting mean levels and the other presenting median levels (i.e., a nonparametric analysis). Within each table, the Mann-Whitney p-value and the normal value v. abnormal value t-Test value are presented for each metabolite. Additionally, the standard deviation (S.D.) is presented for each metabolite in the tables that present the mean levels of the metabolites rather than the median levels.

Table 1 illustrates a typical metabolic profile for the mean level of fatty acids within a population of Down Syndrome patients and a population of normal patients:

TABLE 1

| Fatty Acid | | Normal | | Down Syndrome | |
|---|---|---|---|---|---|
| Compound | t-Test | Mean | S.D. | Mean | S.D. |
| LAURIC ACID | 0.1874 | 0.075 | 0.2897 | 0.3260 | 0.8608 |
| MYRISTIC ACID | 0.5022 | 2.2875 | 3.0146 | 3.0434 | 4.8286 |
| PALMITOLEIC ACID | 0.8122 | 0.0712 | 0.2428 | 0.0869 | 0.2559 |
| PALMITIC ACID | 0.6203 | 23.0625 | 30.183 | 18.7173 | 34.919 |
| LINOLEIC ACID | 0.6124 | 5.45 | 7.1313 | 7.7173 | 20.4982 |
| LINOLENIC ACID | 0.4651 | 3.3775 | 5.8258 | 5.4956 | 12.9802 |
| OLEIC ACID | 0.6296 | 39.8 | 65.011 | 30.4130 | 78.4342 |
| STEARIC ACID | 0.6709 | 3.1125 | 3.6837 | 2.6739 | 4.0469 |
| ARACHIDONIC ACID | 0.7258 | 0.5675 | 2.3453 | 0.4086 | 1.2291 |
| EICOSAPENTAENOIC | 0.7013 | 0.1237 | 0.3415 | 0.1 | 0.1422 |

Table 2 illustrates a typical metabolic profile for the median level of fatty acids within a population of Down Syndrome patients and a population of normal patients:

TABLE 2

| Fatty Acid | | Normal | Down Syndrome |
|---|---|---|---|
| Compound | p-value | Median | Median |
| LAURIC ACID | 0.049 | 0 | 0 |
| MYRISTIC ACID | 0.575 | 1.25 | 1.5 |
| PALMITOLEIC ACID | 0.576 | 0 | 0 |
| PALMITIC ACID | 0.342 | 11.75 | 10.5 |
| LINOLEIC ACID | 1 | 2.25 | 2.5 |
| LINOLENIC ACID | 0.91 | 0.675 | 0.8 |
| OLEIC ACID | 0.391 | 11.5 | 10 |
| STEARIC ACID | 0.564 | 1.5 | 1.5 |
| ARACHIDONIC ACID | 0.103 | 0 | 0 |
| EICOSAPENTAENOIC | 0.361 | 0 | 0 |

Table 3 illustrates a typical metabolic profile for the mean level of simple sugars within a population of Down Syndrome patients and a population of normal patients:

TABLE 3

| Simple Sugars | | Normal | | Down Syndrome | |
|---|---|---|---|---|---|
| Compound | t-Test | Mean | S.D. | Mean | S.D. |
| THREITOL | 0.7620 | 7.8875 | 4.6979 | 7.5217 | 4.5263 |
| ERYTHRITOL | 0.1094 | 6.9875 | 5.3594 | 4.5869 | 5.7537 |
| ARABINOSE | 0.5616 | 0.6375 | 0.6503 | 0.7391 | 0.6719 |
| FUCOSE | 0.2285 | 0.3975 | 1.1847 | 0.1630 | 0.2001 |
| RIBOSE | 0.3449 | 0.6425 | 0.3888 | 0.7652 | 0.5394 |
| ERYTHRITOL.2 | 0.2743 | 9.3625 | 5.3011 | 11.1956 | 6.8286 |
| FRUCTOSE | 0.5252 | 27.1 | 43.9131 | 17.7391 | 61.5203 |
| GLUCOSE mg/dL | 0.7164 | 21.3 | 24.4613 | 19.1739 | 20.8744 |
| GALACTOSE | 0.5750 | 1856.16 | 2477.33 | 1545.69 | 1856.03 |
| MANNOSE | 0.2175 | 3.7625 | 3.2874 | 2.8913 | 2.2409 |
| N-AC-GLUCOSAMINE | 0.5290 | 0.3225 | 0.4437 | 0.2652 | 0.2740 |
| LACTOSE | 0.4813 | 0.625 | 0.9919 | 0.8043 | 0.9503 |
| MALTOSE | 0.3696 | 0.6125 | 1.0830 | 0.8913 | 1.2243 |
| XYLITOL | 0.7618 | 6.465 | 8.2362 | 5.9804 | 4.3894 |
| ARABINITOL | 0.1907 | 0.4862 | 1.5326 | 0.1586 | 0.2097 |
| RIBITOL | 0.9760 | 0.195 | 0.4903 | 0.1978 | 0.2524 |
| ALLOSE | 0.3697 | 0.7512 | 1.7308 | 0.4869 | 0.4873 |
| GLUCURONIC ACID | 0.3784 | 59.7912 | 137.818 | 36.8978 | 66.2477 |
| GALACTONIC ACID | 0.9373 | 149.55 | 776.895 | 162.347 | 507.218 |
| GLUCONIC ACID | 0.8097 | 0.3587 | 0.7182 | 0.3913 | 0.3466 |
| GLUCARIC | 0.3784 | 0.06 | 0.0968 | 0.0913 | 0.1512 |
| MANNITOL | 0.8416 | 45.9612 | 120.018 | 50.9195 | 75.9496 |
| DULCITOL | 0.4140 | 0.1312 | 0.6863 | 0.0413 | 0.0468 |
| SORBITOL | 0.3269 | 0.15 | 0.1870 | 0.2086 | 0.2452 |
| INOSITOL | 0.9174 | 26.8425 | 26.0067 | 27.4782 | 21.6581 |
| SUCROSE | n/a | 0 | 0 | 0 | 0 |
| METANEPHRINE mg/DL | 0.3281 | 0 | 0 | 0.0021 | 0.0104 |

Table 4 illustrates a typical metabolic profile for the median level of simple sugars within a population of Down Syndrome patients and a population of normal patients:

TABLE 4

| Simple Sugars | | Normal | Down Syndrome |
|---|---|---|---|
| Compound | p-value | Median | Median |
| THREITOL | 0.627 | 7.25 | 6.5 |
| ERYTHRITOL | 0.057 | 7 | 1.5 |
| ARABINOSE | 0.504 | 0.5 | 0.5 |
| FUCOSE | 0.192 | 0.05 | 0.1 |
| RIBOSE | 0.663 | 0.675 | 0.65 |
| ERYTHRITOL.2 | 0.431 | 8 | 8.5 |
| FRUCTOSE | 0.102 | 1.5 | 1.5 |
| GLUCOSE mg/dL | 0.716 | 12.5 | 11.5 |
| GALACTOSE | 0.498 | 1056.5 | 692 |
| MANNOSE | 0.352 | 3 | 3 |
| N-AC-GLUCOSAMINE | 0.317 | 0.1 | 0.2 |
| LACTOSE | 0.204 | 0.5 | 0.5 |
| MALTOSE | 0.231 | 0 | 0.5 |
| XYLITOL | 0.602 | 4.3 | 5.05 |
| ARABINITOL | 0.455 | 0.1 | 0.1 |
| RIBITOL | 0.099 | 0 | 0.1 |
| ALLOSE | 0.769 | 0.3 | 0.35 |
| GLUCURONIC ACID | 0.797 | 4.65 | 1.8 |
| GALACTONIC ACID | 0.853 | 8 | 9 |
| GLUCONIC ACID | 0.085 | 0.175 | 0.35 |
| GLUCARIC | 0.471 | 0 | 0.05 |
| MANNITOL | 0.264 | 0.325 | 0.7 |
| DULCITOL | 0.053 | 0 | 0.05 |
| SORBITOL | 0.336 | 0.1 | 0.15 |
| INOSITOL | 0.462 | 16.75 | 27.5 |
| SUCROSE | 1 | 0 | 0 |
| METANEPHRINE mg/dL | 0.187 | 0 | 0 |

Table 5 illustrates a typical metabolic profile for the mean level of amino acids and glycine conjugates within a population of Down Syndrome patients and a population of normal patients:

TABLE 5

| Amino Acids and | | | | | |
|---|---|---|---|---|---|
| Glycine Conjugates | | Normal | | Down Syndrome | |
| Compound | t-Test | Mean | S.D. | Mean | S.D. |
| PROPIONYL GLY | 0.9948 | 0.2225 | 0.6289 | 0.2217 | 0.2848 |
| BUTYRYL GLYCINE | 0.8212 | 0.2213 | 0.6913 | 0.2565 | 0.5292 |
| HEXANOYL GLYCINE | 0.4152 | 0.3938 | 1.3668 | 0.2087 | 0.3006 |
| PHENYL PROP GLY | 0.7414 | 0.5000 | 2.3993 | 0.3543 | 1.0615 |
| SUBERYL GLYCINE | 0.5090 | 0.0300 | 0.0890 | 0.0457 | 0.0903 |
| ISOVALERYL GLY | 0.9786 | 0.4650 | 1.9601 | 0.4543 | 1.1773 |
| TIGLY GLY | 0.4076 | 0.3625 | 0.7605 | 0.2457 | 0.3447 |
| BETA MET CROT GLY | 0.4827 | 1.2750 | 4.3378 | 0.7696 | 0.9522 |
| GLYCINE | 0.3641 | 276.2250 | 199.0262 | 229.2826 | 193.7927 |
| ALANINE | 0.0337 | 455.3000 | 491.5694 | 202.5870 | 412.2851 |
| SARCOSINE | 0.2933 | 1.0988 | 1.2466 | 0.7826 | 1.0703 |
| BETA-ALANINE | 0.9038 | 17.2700 | 11.8828 | 16.9109 | 10.9422 |
| B-AMINOISOBUTYRIC | 0.2457 | 2.2538 | 2.7931 | 1.6609 | 1.1794 |
| SERINE | 0.2684 | 19.7875 | 17.7825 | 15.1087 | 14.8561 |
| PROLINE | 0.0385 | 318.4875 | 322.0118 | 186.5435 | 172.6874 |
| HYDROXY PROLINE | 0.1647 | 81.1000 | 50.1975 | 105.9565 | 74.7918 |
| HYDROXY LYSINE | 0.9268 | 0.7413 | 1.2271 | 0.7152 | 0.9819 |
| ASPARTIC ACID | 0.1032 | 6.7913 | 5.0833 | 11.1761 | 11.8518 |
| ASPARAGINE | 0.7315 | 0.6338 | 0.7522 | 0.6913 | 0.5618 |
| N-AC ASPARTIC | 0.8949 | 0.1800 | 0.6900 | 0.1978 | 0.3761 |
| ORNITHINE | 0.3084 | 40.8000 | 29.5179 | 32.6522 | 30.6162 |
| GLUTAMIC ACID | 0.9576 | 261.4500 | 204.4625 | 258.6522 | 197.4870 |
| GLUTAMINE | 0.1047 | 1.0250 | 3.0633 | 3.5870 | 6.9407 |
| PIPECOLIC ACID | 0.7858 | 3.4488 | 2.7644 | 3.7065 | 3.9971 |
| LEUCINE | 0.0073 | 231.5000 | 181.3793 | 126.5652 | 117.7761 |
| KETO LEUCINE | 0.8192 | 53.0900 | 39.8797 | 50.0196 | 56.3368 |
| VALINE | 0.0440 | 383.8125 | 302.1702 | 239.1087 | 246.4417 |
| KETO-VALINE | 0.9095 | 13.8475 | 12.2042 | 14.2370 | 13.4663 |
| ISOLEUCINE | 0.0651 | 103.6875 | 86.9055 | 67.2609 | 65.4288 |
| KETO-ISOLEUCINE | 0.7580 | 7.5625 | 7.5049 | 8.2000 | 8.0510 |
| LYSINE | 0.7142 | 366.5500 | 287.9107 | 341.2174 | 247.3257 |
| HISTIDINE | 0.8280 | 14.7000 | 12.2138 | 14.0217 | 11.6643 |
| THREONINE | 0.2161 | 292.3125 | 277.0502 | 217.4348 | 195.7553 |
| HOMOSERINE | 0.5177 | 0.3688 | 0.3549 | 0.4261 | 0.3250 |
| METHIONINE | 0.5314 | 34.8375 | 30.2578 | 40.2609 | 34.2282 |
| CYSTEINE | 0.1124 | 152.9625 | 103.4822 | 212.1957 | 155.5338 |
| HOMOCYSTEINE | 0.0690 | 0.0625 | 0.1436 | 0.1565 | 0.2139 |
| CYSTATHIONINE | 0.2705 | 0.1288 | 0.2287 | 0.2087 | 0.2957 |
| HOMOCYSTINE | 0.7456 | 0.0688 | 0.1846 | 0.0565 | 0.1131 |
| CYSTINE | 0.6096 | 0.3138 | 0.7375 | 0.2457 | 0.3030 |
| PHENYLALANINE | 0.2054 | 144.6500 | 79.8540 | 180.2174 | 117.4317 |
| TYROSINE | 0.7914 | 107.1750 | 98.7430 | 100.6087 | 91.7593 |
| TRYPTOPHAN | 0.9187 | 10.3875 | 10.1908 | 10.1087 | 10.4870 |

Table 6 illustrates a typical metabolic profile for the median level of amino acids and glycine conjugates within a population of Down Syndrome patients and a population of normal patients:

TABLE 6

| Amino Acids and | | | |
|---|---|---|---|
| Glycine Conjugates | | Normal | Down Syndrome |
| Compound | p-value | Median | Median |
| PROPIONYL GLY | 0.108 | 0 | 0.15 |
| BUTYRYL GLYCINE | 0.02 | 0 | 0.1 |
| HEXANOYL GLYCINE | 0.011 | 0 | 0.1 |
| PHENYL PROP GLY | 0.933 | 0 | 0 |
| SUBERYL GLYCINE | 0.148 | 0 | 0 |
| ISOVALERYL GLY | 0.005 | 0 | 0.1 |
| TIGLY GLY | 0.976 | 0.125 | 0.1 |
| BETA MET CROT GLY | 0.41 | 0.175 | 0.55 |
| GLYCINE | 0.357 | 280 | 249.5 |
| ALANINE | 0.069 | 312.25 | 2 |
| SARCOSINE | 0.399 | 0.7 | 0.25 |
| BETA-ALANINE | 1 | 16.025 | 18.25 |
| B-AMINOISOBUTYRIC | 0.937 | 1.35 | 1.4 |
| SERINE | 0.247 | 17.25 | 13 |
| PROLINE | 0.265 | 199.5 | 129 |
| HYDROXY PROLINE | 0.214 | 68.5 | 86.5 |
| HYDROXY LYSINE | 0.552 | 0.225 | 0.3 |
| ASPARTIC ACID | 0.454 | 6.35 | 8 |
| ASPARAGINE | 0.399 | 0.475 | 0.45 |
| N-AC ASPARTIC | 0.006 | 0 | 0.05 |
| ORNITHINE | 0.138 | 34.25 | 24 |
| GLUTAMIC ACID | 0.926 | 212.75 | 249 |
| GLUTAMINE | 0.002 | 0.5 | 1 |
| PIPECOLIC ACID | 0.903 | 2.8 | 2.75 |
| LEUCINE | 0.036 | 221.25 | 93.5 |
| KETO LEUCINE | 0.457 | 49.725 | 36.3 |
| VALINE | 0.085 | 368.5 | 176.5 |
| KETO-VALINE | 0.972 | 11 | 9.9 |
| ISOLEUCINE | 0.123 | 93.5 | 51 |
| KETO-ISOLEUCINE | 0.689 | 6.125 | 6.25 |
| LYSINE | 0.627 | 280.25 | 270 |
| HISTIDINE | 0.732 | 11.5 | 11 |
| THREONINE | 0.265 | 236.75 | 155 |
| HOMOSERINE | 0.405 | 0.3 | 0.4 |
| METHIONINE | 0.638 | 33.35 | 38.7 |
| CYSTEINE | 0.104 | 144 | 200 |
| HOMOCYSTEINE | 0.01 | 0 | 0.1 |
| CYSTATHIONINE | 0.09 | 0.05 | 0.1 |
| HOMOCYSTINE | 0.114 | 0 | 0 |
| CYSTINE | 0.115 | 0.075 | 0.15 |
| PHENYLALANINE | 0.467 | 138 | 150.5 |
| TYROSINE | 0.663 | 66 | 67 |
| TRYPTOPHAN | 0.869 | 6.25 | 4.5 |

Table 7 illustrates a typical metabolic profile for the mean level of neurotransmitters within a population of Down Syndrome patients and a population of normal patients:

TABLE 7

| Neurotransmitters | | Normal | | Down Syndrome | |
|---|---|---|---|---|---|
| Compound | t-Test | Mean | S.D. | Mean | S.D. |
| GABA | 0.9990 | 0.8063 | 0.7186 | 0.8065 | 0.8286 |
| HOMOVANILLIC ACID | 0.5579 | 0.1950 | 0.3004 | 0.2609 | 0.4824 |
| NORMETANEPHRINE | 0.0388 | 0.0013 | 0.0079 | 0.0391 | 0.0825 |
| VANILLYLMANDELIC | 0.0849 | 0.0038 | 0.0175 | 0.0152 | 0.0279 |
| METANEPHRINE | 0.5954 | 0.6488 | 2.2671 | 0.4022 | 1.3971 |
| 5-HIAA | 0.5821 | 0.0100 | 0.0258 | 0.0065 | 0.0229 |
| MHPG | 0.5438 | 0.0038 | 0.0175 | 0.0065 | 0.0172 |
| ETHANOLAMINE | 0.6039 | 63.3375 | 46.5176 | 56.7826 | 48.7374 |

Table 8 illustrates a typical metabolic profile for the median level of neurotransmitters within a population of Down Syndrome patients and a population of normal patients:

TABLE 8

| Neurotransmitters | | Normal | Down Syndrome |
|---|---|---|---|
| Compound | p-value | Median | Median |
| GABA | 0.539 | 0.75 | 0.45 |
| HOMOVANILLIC ACID | 0.663 | 0.1 | 0.15 |
| NORMETANEPHRINE | 0.001 | 0 | 0 |
| VANILLYLMANDELIC | 0.019 | 0 | 0 |
| METANEPHRINE | 0.707 | 0.075 | 0.1 |
| 5-HIAA | 0.489 | 0 | 0 |
| MHPG | 0.279 | 0 | 0 |
| ETHANOLAMINE | 0.753 | 65.5 | 65 |

Table 9 illustrates a typical metabolic profile for the mean level of nutritionals within a population of Down Syndrome patients and a population of normal patients:

TABLE 9

| Nutritionals | | Normal | | Down Syndrome | |
|---|---|---|---|---|---|
| Compound | t-Test | Mean | S.D. | Mean | S.D. |
| FORMIMINOGLUTAMIC ACID | 0.0033 | 1.4877 | 0.0462 | 0.8829 | 0.7441 |
| 4-PYRIDOXIC ACID | 0.4924 | 1.2863 | 1.8158 | 1.0370 | 1.0489 |
| PANTOTHENIC ACID | 0.8733 | 299.3500 | 527.7872 | 278.8043 | 466.6548 |
| XANTHURENIC ACID 407 | 0.3163 | 0.0400 | 0.2214 | 0.0043 | 0.0144 |
| KYNURENINE | 0.8218 | 1.2075 | 1.6656 | 1.1283 | 1.1077 |
| QUINOLINIC | 0.8759 | 0.1913 | 1.0503 | 0.2261 | 0.7077 |
| OROTIC ACID | 0.1702 | 0.0133 | 0.0502 | 0.0543 | 0.1344 |
| D-AM LEVULINIC | 0.1741 | 11.3150 | 23.8991 | 5.8217 | 6.0014 |
| 3-METHYL HISTIDINE | 0.8620 | 1.6000 | 1.9553 | 1.5217 | 1.5556 |
| NIACINAMIDE | 0.8650 | 0.8825 | 4.2032 | 0.7587 | 1.3854 |
| PSEUDOURIDINE | 0.7538 | 0.2875 | 0.4220 | 0.3261 | 0.4910 |
| 2-DEOXYTETRONIC | 0.2681 | 1.2250 | 1.1033 | 1.5435 | 1.0757 |
| P-HO-PHEN-ACETIC | 0.5254 | 0.1125 | 0.2399 | 0.1522 | 0.2352 |
| XANTHINE | 0.6903 | 0.0488 | 0.1508 | 0.0630 | 0.1272 |
| UROCANIC ACID | 0.9655 | 0.0625 | 0.3240 | 0.0652 | 0.1722 |
| ASCORBIC ACID | 0.3415 | 0.1000 | 0.4961 | 0.0217 | 0.1043 |
| GLYCEROL | 0.4712 | 36.6663 | 28.3624 | 31.4870 | 26.5956 |

Table 10 illustrates a typical metabolic profile for the median level of nutritionals within a population of Down Syndrome patients and a population of normal patients:

TABLE 10

| Nutritionals | | Normal | Down Syndrome |
|---|---|---|---|
| Compound | p-value | Median | Median |
| FORMIMINOGLUTAMIC | 0.007 | 1.3425 | 0.9119 |
| ACID | | | |
| 4-PYRIDOXIC ACID | 0.839 | 0.8 | 0.8 |
| PANTOTHENIC ACID | 0.932 | 149.5 | 151.5 |
| XANTHURENIC ACID 407 | 0.822 | 0 | 0 |
| KYNURENINE | 0.303 | 0.425 | 0.8 |
| QUINOLINIC | 0.199 | 0 | 0 |
| OROTIC ACID | 0.189 | 0 | 0 |
| D-AM LEVULINIC | 0.587 | 2.85 | 2.6 |
| 3-METHYL HISTIDINE | 0.712 | 1 | 1 |
| NIACINAMIDE | 0.044 | 0 | 0.25 |
| PSEUDOURIDINE | 0.775 | 0 | 0 |
| 2-DEOXYTETRONIC | 0.214 | 1 | 1.5 |
| P-HO-PHEN-ACETIC | 0.39 | 0 | 0 |
| XANTHINE | 0.653 | 0 | 0 |
| UROCANIC ACID | 0.279 | 0 | 0 |
| ASCORBIC ACID | 0.877 | 0 | 0 |
| GLYCEROL | 0.539 | 35.525 | 31.85 |

Table 11 illustrates a typical metabolic profile for the mean level of organic acids within a population of Down Syndrome patients and a population of normal patients:

TABLE 11

| Organic Acids | | Normal | | Down Syndrome | |
|---|---|---|---|---|---|
| Compound | t-Test | Mean | S.D. | Mean | S.D. |
| LACTIC ACID uM/L | 0.3580 | 7455.3375 | 6476.7984 | 6096.8478 | 5027.6119 |
| PYRUVIC ACID | 0.0563 | 16.1375 | 22.5374 | 7.9783 | 10.5760 |
| GLYCOLIC ACID | 0.3684 | 25.2375 | 65.5558 | 43.5435 | 82.6529 |
| ALPHA-OH-BUTYRIC | 0.0965 | 21.4750 | 22.4214 | 11.6304 | 22.0337 |
| OXALIC ACID | 0.0239 | 12.7000 | 21.4652 | 34.8913 | 41.5667 |
| 4-OH-BUTYRIC | 0.4037 | 0.1000 | 0.4114 | 0.1957 | 0.4457 |
| HEXANOIC ACID | 0.9894 | 11.1375 | 21.0619 | 11.0652 | 20.5234 |
| 5-HYDROXYCAPROIC | 0.5482 | 0.2100 | 0.9129 | 0.3239 | 0.5825 |
| OCTANOIC | 0.9308 | 0.6625 | 1.8271 | 0.6304 | 1.0894 |
| BETA-LACTATE | 0.0313 | 11.9625 | 13.0852 | 6.1522 | 7.8327 |
| SUCCINIC ACID | 0.7615 | 1.2125 | 1.5603 | 1.3261 | 1.3366 |
| GLUTARIC ACID | 0.3675 | 0.5588 | 2.2167 | 0.2261 | 0.4970 |
| 2-OXO-GLUTARATE | 0.1486 | 6.1250 | 13.4273 | 11.9130 | 15.8592 |
| FUMARIC | 0.8900 | 0.1275 | 0.3629 | 0.1174 | 0.2146 |
| MALEIC | 0.0986 | 2.7875 | 3.1928 | 4.7348 | 4.9316 |
| MALIC ACID | 0.4096 | 2.0000 | 2.7087 | 2.5587 | 2.4817 |
| ADIPIC ACID | 0.8252 | 0.1375 | 0.5126 | 0.1174 | 0.1940 |
| SUBERIC ACID | 0.7434 | 0.1925 | 0.3964 | 0.2196 | 0.2557 |
| SEBACIC ACID | 0.0816 | 0.6875 | 0.8276 | 0.3413 | 0.6933 |
| GLYCERIC ACID | 0.6070 | 28.0000 | 22.2955 | 25.2609 | 18.9406 |
| BETA-OH-BUTYRIC | 0.4624 | 61.4750 | 53.3212 | 50.2174 | 60.5152 |
| METHYLSUCCINIC | 0.1623 | 0.1775 | 0.7137 | 0.5087 | 0.9728 |
| METHYLMALONIC | 0.9946 | 25.3988 | 23.5841 | 25.4348 | 18.3025 |
| ETHYLMALONIC | 0.3967 | 28.3250 | 65.1132 | 16.7174 | 42.6016 |
| HOMOGENTISIC ACID | 0.3281 | 0.0138 | 0.0519 | 0.0370 | 0.1047 |
| PHENYLPYRUVIC ACID | 0.0254 | 0.0375 | 0.0749 | 0.1435 | 0.2063 |
| SUCCINYLACETONE | 0.9787 | 0.2600 | 0.5466 | 0.2630 | 0.3549 |
| 3-OH-ISOVALERIC | 0.5760 | 3.5813 | 3.9367 | 3.0652 | 3.2301 |
| PHOSPHATE mg/dL | 0.6839 | 1.0363 | 1.2176 | 0.9174 | 1.0422 |
| CITRIC ACID | 0.9542 | 36.7000 | 47.5228 | 37.3261 | 37.4884 |
| HIPPURIC ACID | 0.0613 | 21.4125 | 24.8725 | 12.9783 | 9.6218 |
| URIC ACID mg/dL | 0.5409 | 0.4200 | 0.7250 | 0.3391 | 0.3086 |

Table 12 illustrates a typical metabolic profile for the median level of organic acids within a population of Down Syndrome patients and a population of normal patients:

TABLE 12

| Organic Acids | | Normal | Down Syndrome |
|---|---|---|---|
| Compound | p-value | Median | Median |
| LACTIC ACID uM/L | 0.521 | 5563.5 | 5138 |
| PYRUVIC ACID | 0.311 | 5.25 | 5 |
| GLYCOLIC ACID | 0.668 | 12.5 | 13 |
| ALPHA-OH-BUTYRIC | 0.046 | 17.25 | 1 |
| OXALIC ACID | 0.007 | 5 | 23.5 |
| 4-OH-BUTYRIC | 0.103 | 0 | 0 |
| HEXANOIC ACID | 0.731 | 6.25 | 4 |
| 5-HYDROXYCAPROIC | 0.009 | 0 | 0.05 |
| OCTANOIC | 0.643 | 0 | 0 |
| BETA-LACTATE | 0.198 | 6 | 4.5 |
| SUCCINIC ACID | 0.433 | 0.5 | 1 |
| GLUTARIC ACID | 0.095 | 0 | 0.05 |
| 2-OXO-GLUTARATE | 0.034 | 1 | 5 |
| FUMARIC | 0.175 | 0 | 0.05 |
| MALEIC | 0.108 | 1.525 | 2.5 |
| MALIC ACID | 0.252 | 0.975 | 1.6 |
| ADIPIC ACID | 0.009 | 0 | 0.05 |
| SUBERIC ACID | 0.162 | 0.05 | 0.1 |
| SEBACIC ACID | 0.13 | 0.35 | 0.1 |
| GLYCERIC ACID | 0.814 | 25.25 | 26 |
| BETA-OH-BUTYRIC | 0.414 | 66.25 | 33 |
| METHYLSUCCINIC | 0.002 | 0 | 0.15 |
| METHYLMALONIC | 0.563 | 21.3 | 24.3 |
| ETHYLMALONIC | 0.407 | 6.125 | 2.8 |
| HOMOGENTISIC ACID | 0.218 | 0 | 0 |
| PHENYLPYRUVIC ACID | 0.017 | 0 | 0.05 |
| SUCCINYLACETONE | 0.188 | 0.05 | 0.1 |
| 3-OH-ISOVALERIC | 0.881 | 2.375 | 1.45 |
| PHOSPHATE mg/dL | 0.949 | 0.65 | 0.7 |
| CITRIC ACID | 0.38 | 13 | 22.5 |
| HIPPURIC ACID | 0.287 | 11.25 | 9 |
| URIC ACID mg/dL | 0.426 | 0.2 | 0.25 |

If a metabolic profile of a specific patient reflected the same profile as the Down Syndrome group, the method of the present invention could be used to diagnose and suggest treatment for this patient. In the example profile data, formiminoglutamate (FIGLU) in Down Syndrome (Mean=0.8829, Median=0.9119) is decreased from normal (Mean=1.4877, Median=1.3425). (See Tables 9 and 10). FIGLU is a metabolite of histidine, which contributes a mono-carbon to tetrahydrofolate. In folate deficiency, FIGLU accumulates because there is little folate to accept the mono-carbon. Therefore, folate deficiency results in a shortage of mono-carbon tetrahydrofolate. Additionally, mono-carbons are necessary to synthesize biologically important molecules. Mono-carbon tetrahydrofolate is a major source of mono-carbons used in cellular biosynthesis. Mono-carbons are required to synthesize purines (e.g. adenine & guanine), components of DNA, RNA, and other important molecules. In Down Syndrome, purine synthesis is accelerated. There are signs of mono-carbon shortage but folate is not deficient. In our data, we see that FIGLU is reduced. This probably reflects an increased demand for mono-carbons due to accelerated purine synthesis. Therefore, a treatment of mono-carbon supplements could be prescribed for this patient.

Another metabolite that should be addressed according to this data is normetanephrine. (See Tables 7 and 8). Normetanephrine is a metabolite of the neurotransmitter norepinephrine (NE). NE requires a mono-carbon to be metabolized to epinephrine. Mono-carbon shortage explains the finding of elevated normetanephrine in Down Syndrome (Mean=0.039, Median=0) compared to normal amniotic fluid (Mean=0.0013, Median=0).

Cystathionine beta synthase (CBS) is an enzyme whose gene is on chromosome 21. This enzyme catalyzes the reaction of serine and homocysteine to cystathionine. Subsequently cystathionine is converted to cysteine. Since this enzyme will be present in excess in a Down Syndrome patient, we would expect a reduction in the substrates serine and homocysteine and an increase in the products cystathionine and cysteine. Referring to Tables 5 and 6, serine in a Down Syndrome patient (Mean=15.1087, Median=13) is reduced from levels in a normal patient (Mean=19.7875, Median=17.25). Cystathionine is greater in a Down Syndrome patient (Mean=0.2087, Median=0.1) than in a normal patient (Mean=0.1288, Median=0.05). Cysteine is greater in a Down Syndrome patient (Mean=212.1957, Median=200) than in a normal patient (Mean=152.9625, Median=144). Homocysteine is greater in a Down Syndrome patient (Mean=0.1565, Median=0.1) than in a normal patient (Mean=0.0625, Median=0.0).

Prior to the method of the current invention, the aforementioned results would ordinarily be difficult to comprehend. However, pursuant to the invention, the examination of the results from the nutritional molecule FIGLU and from the neurotransmitter normetanephrine enables a treatment to be suggested for these conditions. These results indicate a shortage of mono-carbons. Homocysteine would normally be methylated to methionine and then converted to s-adenosyl-methionine (SAM). SAM is a major source of mono-carbons in the cell. Since such mono-carbons are in short supply, homocysteine accumulates due to the lack of mono-carbons necessary to methylate homocysteine to methionine. Again, this suggests that a supplement of mono-carbons should be prescribed. Thus, a treatment regimen that supplements mono-carbons is prescribed to a patient pursuant to the data provided by the broad metabolic profile analysis described herein.

Holocarboxylase synthase is located on chromosome 21. This enzyme causes metabolic activation of the vitamin biotin. The data here suggest that some biotin dependent enzymes are accelerated in Down Syndrome. First, beta-lactate, also known as 3-hydroxy-propionate, is a marker for propionyl COA carboxylase, a biotin dependent enzyme. Beta-lactate in a Down Syndrome patient (Mean=6.1522, Median=4.5) is lower than in a normal patient (Mean=11.9625, Median=6). (See Tables 11 and 12). Additionally, leucine is lower in a Down Syndrome patient (Mean=126.5652, Median=93.5) than in a normal patient (Mean=231.5, Median=221.25). (See Tables 5 and 6). Isoleucine is also lower in a Down Syndrome patient (Mean=67.2609, Median=51) than in a normal patient (Mean=103.6875, Median=93.5). Catabolism of both isoleucine and leucine require biotin.

These results suggest that patients with Down Syndrome have an acceleration of biotin-dependent pathways due to an increase in gene dosage from holocarboxylase synthase. Biotin is generally thought to be non-toxic. If these accelerated pathways were harmful, a patient may benefit from a biotin restriction pursuant to the aforementioned data.

Another treatment that is suggested by the method of globally analyzing a complete metabolic profile of this patient for chromosomal abnormalities is a tetra-hydra-biopterin supplement. Phenylpyruvate (phenylpyruvic acid) is elevated in a Down Syndrome patient (Mean=0.1435, Median=0.05) over a normal level (Mean=0.0375, Median=0). (See Tables 11 and 12). Phenylalanine is increased in a Down Syndrome patient (Mean=180.2174, Median=150.5) over a normal level (Mean=144.65, Median=138). (See Tables 5 and 6). These two metabolites are elevated in phenylketonuria (PKU), a disease which causes mental retardation. The enzyme affected is phenylalanine hydroxylase. The enzyme requires tetrahydrabiopterin, thought to be deficient in Down Syndrome patients. The aforementioned data suggests that tetrahydrabiopterin deficiency is present in Fetal Down Syndrome and should be treated by prescribing tetrahydrabiopterin supplements.

Furthermore, analysis of the metabolite data may suggest a vitamin B6 supplement. Turning again to Tables 11 and 12, oxalic acid (oxalate) in a Down Syndrome patient (Mean=34.8913, Median=23.5) is elevated over normal levels (Mean=12.7, Median=5). This characteristic suggests a functional deficiency of vitamin B6, also known as pyridoxine. Therefore, a vitamin B6 supplement should be prescribed.

The above findings can be assembled to give an itemized biochemical characterization of fetal Down Syndrome. Some of the biochemical abnormalities are treatable. As shown below, several types of treatment may be prescribed based on the biochemical characterization of Down Syndrome discussed above.

| ABNORMALITY | PRESCRIBED SUPPLEMENT(S) |
|---|---|
| Mono-carbon Shortage | Folate, B12, Mono-carbon donors (ex. |
| | Methionine, Betaine, Choline, |
| | Dimethylglycine) |
| Increased Homocysteine | Folate, B12, Mono-carbon donors (ex. |
| | Methionine, Betaine, Choline, |
| | Dimethylglycine) |
| Increased NE | Folate, B12, Mono-carbon donors (ex. |
| | Methionine, Betaine, Choline, |
| | Dimethylglycine) |
| Functional B6 deficiency | B6 |
| Reduced serine | Serine |
| Tetra-hydro-biopterin deficient | Tetra-hydro-biopterin |

With the current method, a simultaneous assessment of all metabolites improves the assessment of individual metabolites and suggests treatment that might have otherwise been overlooked.

The particular examples set forth herein are instructional and should not be interpreted as limitations on the applications to which those of ordinary skill are able to apply this invention. Modifications and other uses are available to those skilled in the art which are encompassed within the spirit of the invention as defined by the scope of the following claims.

What is claimed is:

1. A method of identifying an abnormal metabolite profile in a fetus and comparing the profile to a metabolic profile of Down Syndrome, comprising:
a) obtaining an amniotic fluid specimen by placing a syringe having a needle into a uterus and withdrawing the amniotic fluid specimen via the needle,
b) identifying a quantity for each metabolite that is present in the amniotic fluid specimen using a gas chromatograph/mass spectrometer,
c) compiling a profile of the amniotic fluid specimen that lists each metabolite and the quantity for each metabolite,
d) comparing the amniotic fluid specimen profile with a control profile representative of normal levels of each metabolite in amniotic fluid by comparing the quantity of each metabolite,
e) identifying a plurality of abnormal metabolite levels in the amniotic fluid specimen when the comparing step of step d) reveals that the profile of metabolites a pattern of the quantity of each metabolite in the amniotic fluid specimen compiled in step c) differs from the control profile, and a pattern in the quantity of each metabolites in the control profile, and
f) comparing the plurality of abnormal metabolite levels to the metabolic profile of Down Syndrome.

2. The method of claim 1, wherein the identifying step comprises revealing that a metabolite selected from the group consisting of: formiminoglutamate, normetanephrine, homocysteine, oxalic acid, serine, and tetra-hydro-biopterin and combinations thereof in the amniotic fluid specimen differs in quantity from the control profile.

3. The method of claim 2, wherein the quantity for each metabolite listed by the control profile comprises a mean level.

4. The method of claim 2, wherein the quantity for each metabolite listed by the control profile comprises a median level.

5. The method of claim 2, further comprising, after the obtaining an amniotic fluid specimen step, storing the amniotic fluid specimen at around −20° C.

6. A method of identifying abnormal metabolites in a fetus, comprising:
obtaining an amniotic fluid specimen by placing a needle into a uterus and withdrawing the amniotic fluid specimen via the needle,
identifying a quantity for each metabolite that is present in the amniotic fluid specimen by analyzing the amniotic fluid specimen using a gas chromatograph/mass spectrometer,
compiling a profile of the amniotic fluid specimen, wherein the profile lists each metabolite and the quantity for each respective metabolite present in the amniotic fluid specimen,
obtaining a control profile, wherein the control profile lists a quantity for each metabolite present in the amniotic fluid specimen for a control population without Down Syndrome,
identifying a plurality of abnormal quantities of metabolites of the profile of the amniotic fluid specimen by comparing the quantity of each metabolite with the quantity for that respective metabolite of the control profile, and
comparing the identified plurality of abnormal quantities of metabolites to a metabolic profile of Down Syndrome.

7. The method of claim 6, wherein the step of identifying a plurality of abnormal quantities of metabolites comprises identifying decreased concentration of formiminoglutamic acid, increased concentration of homocysteine, increased concentration of normetanephrine, decreased concentration of oxalic acid, decreased concentration of serine, and decreased concentration of tetra-hydro-biopterin and combinations thereof.

8. The method of claim 6, wherein the step of identifying a plurality of abnormal quantities of metabolites is comprised of identifying at least two abnormal quantities chosen from the group consisting of decreased concentration of formiminoglutamic acid, increased concentration of homocysteine, increased concentration of normetanephrine, decreased concentration of oxalic acid, decreased concentration of serine, and decreased concentration of tetra-hydro-biopterin and combinations thereof.

9. The method of claim 1, wherein the step of identifying the quantity of each metabolite comprises identifying the quantity of formiminoglutumate and oxalic acid.
